(19) United States
(12) Patent Application Publication
(10) Pub. No.: US 2011/0301633 A1
(43) Pub. Date: Dec. 8, 2011

(54) CONTROLLED RELEASE MECHANISM FOR BLOOD VESSEL FILTRATION DEVICE

(76) Inventors: Patrick Muck, Crestview Hills, KY (US); Axel Thors, Ludlow, KY (US); Kevin Harper, Mason, OH (US)

(21) Appl. No.: 12/961,413

(22) Filed: Dec. 6, 2010

Related U.S. Application Data
(60) Provisional application No. 61/266,986, filed on Dec. 4, 2009.

(51) Int. Cl. A61F 2/01

(52) U.S. Cl. 606/200

(57) ABSTRACT

A filtration device is disclosed having a body configured for placement within a portion of the vasculature of a patient, where the body includes a plurality of threads, each thread having a proximal end and a distal end. The filtration device further includes an cap forming an apex, wherein at least one of the cap and the body include a selectively transformative material. The filtration device has a first configuration, where the cap substantially retains the distal end for each of the plurality of threads at the apex such that the filter device is substantially operable and a second configuration, where the filtration device is substantially inoperable as a result of at least one of the body and the cap being selectively transformed by electromagnetic induction. Systems and methods involving such filtration devices are also disclosed herein.

### PRIORITY

This application claims priority to and benefit of U.S. Provisional application No. 61/266,986, entitled “Controlled Release Mechanism For Blood Vessel Filtration Device”, filed on Dec. 4, 2009, which is herein incorporated by reference in its entirety.

### TECHNICAL FIELD

Embodiments of the present invention relate, in general, to a filtration device for a blood vessel, and, in particular, to a system and method of causing a filtration device to transform in vivo.

### BACKGROUND

Deep vein thrombosis and pulmonary embolism are increasingly common medical conditions that may contribute substantially to individual patient morbidity and mortality as well as global healthcare costs. Deep vein thrombosis develops within the deep veins of the lower extremities but also can involve or arise solely from the veins of the pelvis or the upper extremities. When blood clots float from a large vein in the lower body through the vena cava to the heart and lungs they may cause a pulmonary embolism. Although the natural processes of the body may operate to eliminate such emboli, they tend to be rather slow. If an embolus is large enough and migrates to the lung of a person, the result may be catastrophic or fatal (e.g., shortness of breath, hypoxia, heart failure, hypotension, myocardial infarction secondary to right heart strain).

To prevent such a pulmonary embolism, filters have been implanted within a vein, typically the inferior vena cava, to trap a large blood clot before it reaches a potentially dangerous site in a patient's body while continuing to allow blood to pass freely through the filter around the clot. The trapped blood clot may dissolve over time. Filters of various kinds are currently known, and they may be designed to be physically removed after beneficial use or they may be intended for permanent implantation in the patient's body. Complications that may arise from using a blood clot filter, particularly if it is implanted long-term, include recurrent thrombosis at the site of the filter, perforations of the vena cava, migration of the filter, erosion, etc., along with a continued regimen of anti-coagulants. Retrievable filters also may require another invasive procedure to remove the device.

Thus, it may be advantageous to provide a blood clot filter that is easily implantable and can be wholly or partially dissolvable to reduce the likelihood of further complication or harm to a patient. In addition, it may be advantageous to embed a blood clot filter with an anti-coagulant, so the filter may not only impede the migration of a blood clot but also prevent its formation in the first place.

### SUMMARY

In accordance with one embodiment, a filtration device is disclosed having a body configured for placement within a portion of the vasculature of a patient, where the body includes a plurality of threads, each thread having a proximal end and a distal end. The filtration device further includes a cap forming an apex, wherein at least one of the cap and the body include a selectively transformative material. The filtration device has a first configuration, where the cap substantially retains the distal end for each of the plurality of threads at the apex such that the filter device is substantially operable and a second configuration, where the filtration device is substantially inoperable as a result of at least one of the body and the cap being selectively transformed by electromagnetic induction.

In accordance with another embodiment, a filtration system is disclosed including a filtration device having a body configured for placement within a portion of the vasculature of a patient, wherein the body comprises a plurality of threads, each thread having a proximal end and a distal end. The filtration device further includes a cap forming an apex, where at least one of the cap and the body include a selectively transformative material. The filtration device has a first configuration, where the cap substantially retains the distal end for each of the plurality of threads at the apex such that the filtration device is substantially operable, and a second configuration, where the filtration device is substantially inoperable as a result of at least one of the body and the cap being selectively transformed by electromagnetic induction. The filtration system further includes an electromagnetic induction system, the electromagnetic induction system being configured to generate a magnetic field sufficient to selectively change the filtration device from the first configuration to the second configuration.

In accordance with yet another embodiment, a filtration method is disclosed having the steps of providing a filtration device, where the filtration device includes a body configured for placement within a portion of the vasculature of a patient, where the body includes a plurality of threads, each thread having a proximal end and a distal end. The filtration device further includes an cap forming an apex, where at least one of the cap and the body comprises a selectively transformative material The filtration device has a first configuration, where the cap substantially retains the distal end for each of the plurality of threads at the apex such that the filtration device is substantially operable, and a second configuration, where the filtration device is substantially inoperable as a result of at least one of the body and the cap being selectively transformed by electromagnetic induction. The filtration method further includes the steps of providing an electromagnetic induction system, inserting the filtration device into a portion of the vasculature of a patient, and generating a magnetic field with the electromagnetic induction system to selectively transform the filtration device from the first configuration to the second configuration.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings. In the drawings, like numerals represent like elements throughout the several views.

FIG. 1 is a front elevation view of one version of a filtration device.

FIG. 2 is a top plan view of the filtration device of FIG. 1.

FIG. 3 is a sectional view of the filtration device of FIG. 1 shown in a collapsed position within a blood vessel.

FIG. 4 is a sectional view of the filtration device of FIG. 1 shown in an expanded position within a blood vessel.

FIG. 5 is a sectional view of the filtration device of FIG. 1 shown in an expanded position and operable configuration within a blood vessel.

FIG. 6 is a sectional view of the filtration device of FIG. 1 shown in an inoperable configuration within a blood vessel.

FIG. 7 is a front elevation view of an alternative version of a filtration device.

FIG. 8 is a sectional view of the filtration device of FIG. 7 shown in a collapsed or closed position within a blood vessel.

FIG. 9 is a sectional view of the filtration device of FIG. 7 shown in an expanded or operable configuration within a blood vessel.

FIG. 10 is a sectional view of the filtration device of FIG. 7 shown in an expanded or operable configuration within a blood vessel after being selectively transformed to release a cap prior to the filtration device transitioning to an inoperable configuration.

FIG. 11 is a schematic diagram of one version of an induction system.

FIG. 12 is a partial front perspective view of one version of a filtration device having a cap configured to be releasable from a plurality of other filter elements.

FIG. 13 is a partial front perspective view of an alternate version of a filtration device having a cap configured to be releasable from a plurality of other filter elements.

FIG. 14 is a partial rear perspective view of an alternate version of a filtration device having a cap configured to be releasable from a plurality of other filter elements.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive. As used herein the term “superior” will be understood to mean a relative position above or closer to the head, while the term “inferior” will be understood to mean a relative position below or toward the feet. As will be understood by one of ordinary skill in the art, however, the use of such directional and/or positional terms should not be used to limit the present application, unless otherwise indicated.

As shown in the figures, versions of a filtration device may be positioned and deployed within a blood vessel, such as the inferior vena cava, to prohibit the migration of an embolism. The disclosed filtration devices may be utilized for permanent implantation and/or they may be intended to be absorbed, retrieved, or otherwise removed from the body. Alternative versions of a filtration device may be used to control the release of a deployed filter in a non-invasive manner.

### I. Filtration Device

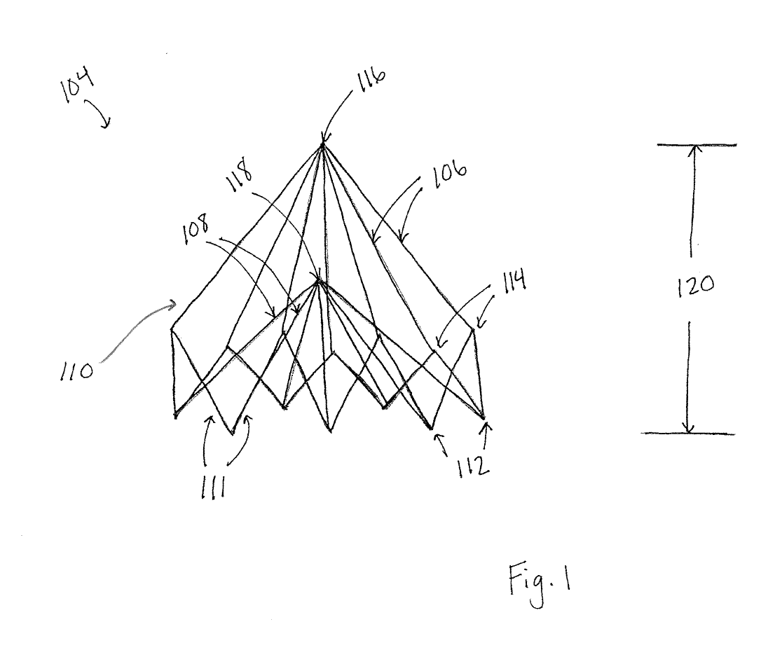

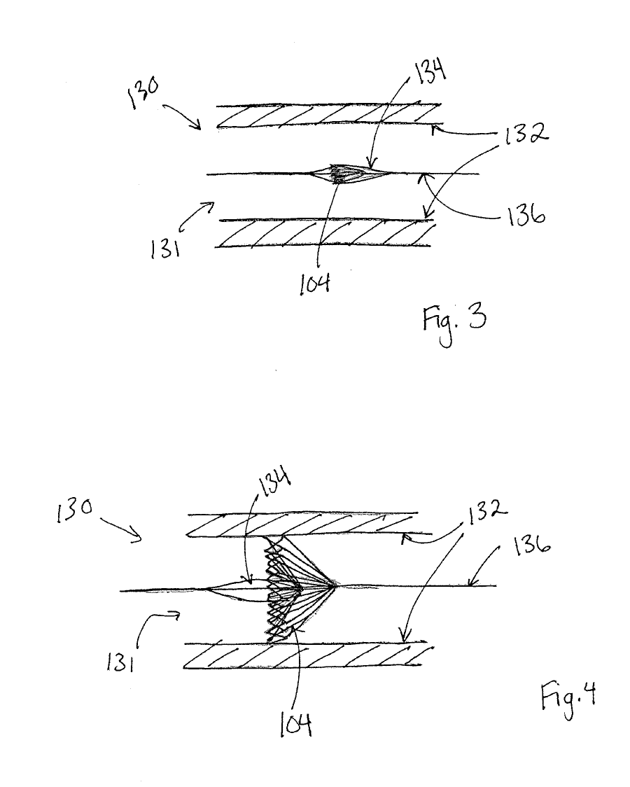

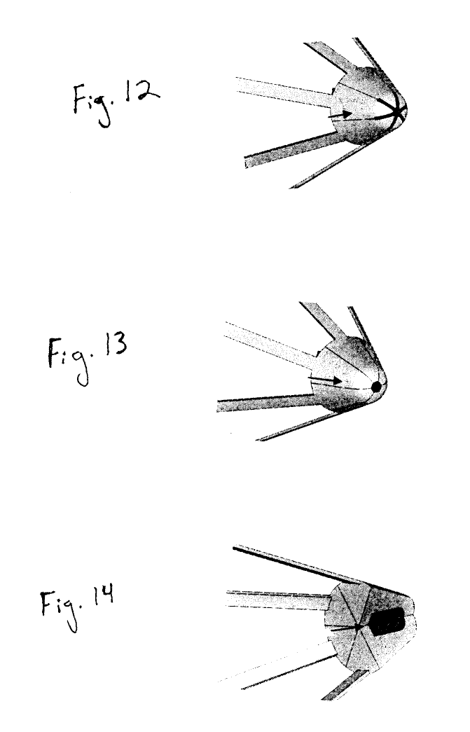

Filtration devices described below are generally configured for use within a blood vessel to impede the movement of emboli and thereby prevent pulmonary embolism, however, other applications, including medical applications, are contemplated. Selected embodiments are hereinafter described in detail in connection with the views and examples of FIGS. 1-14. As shown in FIGS. 1-6, one version of a blood filter device (104) is shown having a flexible body (110), a first set of threads (106), and a second set of threads (108). Body (110) further comprises a plurality of segments (111) that can alternate in a zig-zag or toothed arrangement to generally form a ring having a plurality of inferior nodes (112) and a plurality of superior nodes (114), in which nodes (112, 114) can be formed at the intersection(s) of segments (111). In this way, body (110) may be moveable between a collapsed position (FIG. 3) and an expanded position (FIG. 4). Of course, body (110) may have any other suitable structure to permit operation of device (104) between a collapsed position and an expanded or operable position or configuration as will be appreciated by one of ordinary skill in the art. By way of example, body (110) may comprise two or more sets of segments (111) forming two or more interlocking rings. In other versions, instead of an interlocking or zig-zag pattern, each circumferential blood vessel fixation point, which may be for example at nodes (112, 114) of the filter (104), may work independently and be permanent or absorbable. The body (110) can be, for example, self-expanding or balloon expandable.

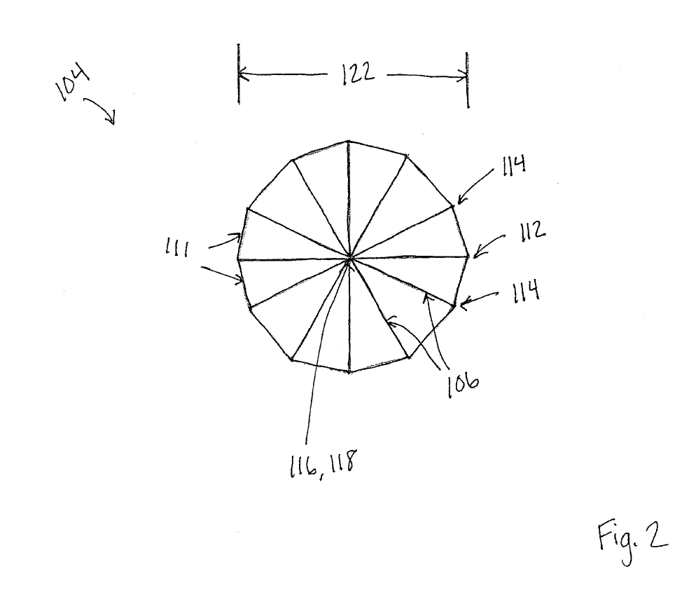

Extending from nodes (112, 114) are the first set of threads (106) and second set of threads (108). The first set of threads (106) extends from the plurality of superior nodes (114) and each thread (106) is joined together at a superior apex (116). Similarly, the second set of threads (108) extends from the plurality of inferior nodes (112) and each thread (108) can be joined together at an inferior apex (118). In this way, when device (104) is in an expanded or operable position, the second set of threads (108) may form a smaller conical shape that nests within a larger conical shape formed from the first set of threads (106). In one version, the dimensions of device (104) can include threads (106, 108) being approximately 24 mm. in length, segments (111) of body (110) being approximately 22 mm. in length, height (120) of device (104) being approximately 46 mm., and width (122) of device (104), in an expanded position, being approximately 36 mm. to approximately 38 mm. Although shown in FIG. 2 as being substantially in radial alignment, it should be understood that threads (106, 108) may be radially offset from each other to increase or change the filtration capabilities of the device (104). Similarly, although shown in FIG. 1 as being substantially in vertical alignment, apices (116, 118) may be vertically offset. It will be appreciated that filtration devices described in accordance with versions herein may have any suitable number, dimension, and/or configuration of threads, nodes, and/or apices.

Threads (106, 108) and/or body (110) may be formed from any suitable biocompatible material as will be understood by one of ordinary skill in the art. For example, threads (106, 108) and body (110) may be absorbable, partially absorbable, non-absorbable, and/or have shape memory characteristics. In versions contemplated herein, body (110) may be formed from a body-tolerated metal or alloy, such as medical grade stainless steel, nickel-titanium alloys, cobalt-chromium-nickel alloys, or cobalt-chromium-nickel-molybdenum-iron alloys. Threads (106, 108) may be formed, for example, from a bio-absorbable flexible material, from synthetic absorbable sutures made primarily of polyglycolic acid, or polydioxanone (PDO, PDS) biodegradable synthetic polymer. Of course, threads (106, 108) and/or body (110) may alternatively be made from a non-dissolving or non-absorbable material such as polytetrafluoroethylene (PTFE). Threads (106, 108) may alternatively be constructed from an absorbable polymer that can be extruded and/or woven. These include, but are not limited to, polylactic acid, polycaprolactone, polyglycolide, poly-3-hydroxybutyrate, polydioxanone, and combinations thereof.

Threads (106, 108) and/or body (110) may also be embedded, impregnated, or injected, or otherwise incorporated with a material to prevent the patient's body from rejecting the device (104) or to further impede embolus migration by expediting the dissolution of emboli or for any other reason. For example, threads (106, 108) and/or body (110) may be embedded with an anti-coagulant, such as heparin, that may be on time-control release to reduce or prevent the formation of emboli and/or expedite the dissolution of emboli. In such an example, an embolus that may be naturally absorbed by the bloodstream over a 30-90 day period may be dissolved more quickly due to an injected material like heparin released from device (104). In another example, threads (106, 108) may be coated with a coating agent, such as a medication, for minimal release into the blood stream. Additionally, the filter (104) components may be inert and non-reactive. The biomaterial and/or polymers from which the filter (104) may be formed may be bonded with anti-coagulants like heparin, which may reduce or prevent clot formation and lengthen device patency. Other agents that could be bonded with the material are anti-platelet agents which may act to inhibit platelet aggregation, which is the first step in the clot formation process.

It should also be appreciated that threads (106, 108) may be joined to the body at the inferior and superior nodes (112, 114), and to each other at the superior and inferior apices (116, 118), in any suitable configuration, which may depend on the chosen material of the threads (106, 108). By way of example, the uniting of the respective sets of threads (106, 108) at their corresponding junctions (112, 114, 116, 118) may be effected by use of an adhesive, by heat fusion, by placement of an cap, by placement of a dissolvable member, by placement of a removable member, or by tying. Threads (106, 108) may also be tapered, such that they are thicker, for example, at the inferior and superior nodes (112, 114) and thinner at the superior and inferior apices (116, 118). Such tapering may ensure that no piece of absorbable material, such as a piece of thread (106, 108) or adhesive, of any dangerous size that potentially breaks off or dissolves from the device (104) can be carried along by the blood flow to a potentially dangerous location such as, for example, the heart, or lung. Of course, threads (106, 108) may be tapered in the opposite fashion such that they are thicker at the superior and inferior apices (116, 118) and thinner at the inferior and superior nodes (112, 114). Or threads (106, 108) may not be tapered at all, having a consistent width along the entire length of the thread (106, 108).

FIGS. 3-6 depict one version of filter device (104) in the lumen (131) of a blood vessel (130), which may be defined by an interior wall (132). Device (104) may be positioned in any suitable location within a person's vascular system as will be appreciated by one of ordinary skill in the art. By way of example, device (104) may be placed in the inferior vena cava, the jugular vein, the brachial vein, or the common femoral vein. Device (104) may also be positioned either upstream or downstream of a junction between the hosting vein and its sizeable branches. It should be understood that placement of device (104) may be performed by any suitable known technique. For instance, while in a collapsed position as shown in FIG. 3, device (104) may be placed through the common femoral, jugular, or large-caliber peripheral veins using the Seldinger technique as is known by those in the art. In the illustrative version, as shown in FIGS. 3-6, device (104) is placed within a blood vessel (130) while device (104) is in a collapsed position as shown in FIG. 3. In one version, device (104) is positioned such that the apices (116, 118) of the device (104) point in the direction of blood flow (142), however, other configurations are contemplated.

Once placed within the lumen (131) of blood vessel (130), device (104) may be deployed from a collapsed position to an expanded or operable position and affixed into place within the vessel (130). In the illustrated version, a balloon (134) and guide wire (136) may be used to selectively actuate the device (104) from a collapsed position to an expanded position. As shown in FIGS. 3-4, balloon (134), located in the interior of the collapsed device (104), may be inflated such that the inflation of balloon (134) causes a corresponding expansion of the device (104) as the circumference of body (110) increases. In the expanded position, device (104) may be affixed to the interior wall (132) of blood vessel (130) in any suitable manner as will be understood by one of ordinary skill in the art. For example, hooks (not pictured), which may be permanent or absorbable, may extend from body (110) of the device (104) to secure the device (104) to a blood vessel wall. Once device (104) is in place within blood vessel (130), balloon (134) may be deflated and removed via guide wire (136).

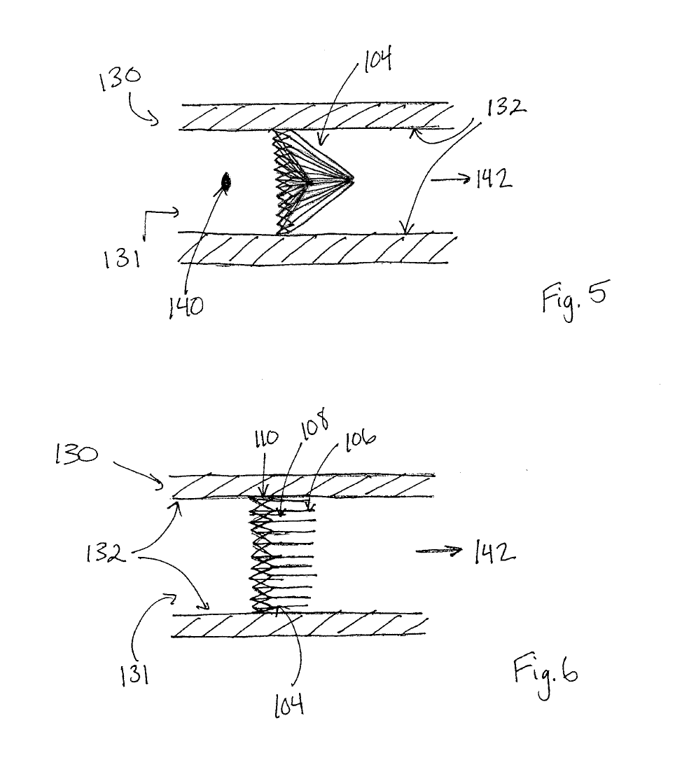

Referring to FIG. 5, device (104) is shown in an expanded position within blood vessel (130). In this way, device (104) is operable to trap an embolus (140) traveling along in the direction (142) of blood flow. For example, as the embolus (140) travels along the blood stream in direction (142), it may encounter one or more threads (106, 108) of device (104). Based on the principles of fluid dynamics, wherein blood flow is fastest near the central axis of a vessel, embolus (140) may be traveling near the center of vessel (130), such that it may contact threads (106, 108) at a point near apices (116, 118) where threads (106, 108) are closely disposed. In this way, embolus (140) may be trapped and impeded from further travel by device (104). If device (104) is embedded with a drug or other material, such as an anti-coagulant, the drug may be released while the device (104) is positioned within the vessel (130). For example, if device (104) is embedded with a heparin anti-coagulant, the heparin may be released and dissolve an embolus (140) trapped by device (104).

Over time, all or a portion of device (104) may be removed from blood vessel (130) or it may remain permanently implanted, for example, in an inoperable configuration as shown in FIG. 6. Whether or not device (104) is removed from vessel (130) may depend on a number of factors such as the materials from which device (104) is manufactured and patient health. Even if device (104) is not removed from blood vessel (130), it may dissolve over time such that at some point it no longer remains in the body. In still other versions, device (104) may only partially dissolve, leaving at least a portion of the device (104) harmlessly implanted in vessel (130). For example, body (110) may be made of a non-dissolvable material such as a medical grade stainless steel or nickel-titanium alloy, and threads (106, 108) may comprise a dissolvable material such as polydioxanone suture material. In this way, over time, threads (106, 108) may dissolve away, leaving behind body (110) which poses no substantial impediment to blood flow. In still another version, as shown in FIG. 6, body (110) and threads (106, 108) may be non-dissolvable while the material uniting the threads at apices (116, 118) may be dissolvable. In this way, over time the uniting material at apices (116, 118) may dissolve away, threads (106, 108) may be attached only at nodes (112, 114), and the remainder of device (104) may harmlessly remain as the disunited threads (106, 108) align themselves with blood flow (142), posing no substantial impediment to the blood flow.

Of course, the above-described filter device (104) is described by way of example only. Any other suitable type of filter device (104) and associated components may be used. By way of example, device (104) may comprise only one set of threads extending from body (110) instead of having two sets of threads (106, 108), one nesting inside the other. In another example, instead of two nested cones formed by threads (106, 108), body (110), apices (116, 118), and nodes (112, 114), filter device (104) may comprise a single cone or three or more cones.

Alternatively, filter device (104) may have any other suitable components, features, configurations, functionalities, operability, etc. Other suitable variations of filter device (104) and associated components will be apparent to those of ordinary skill in the art in view of the teachings herein.

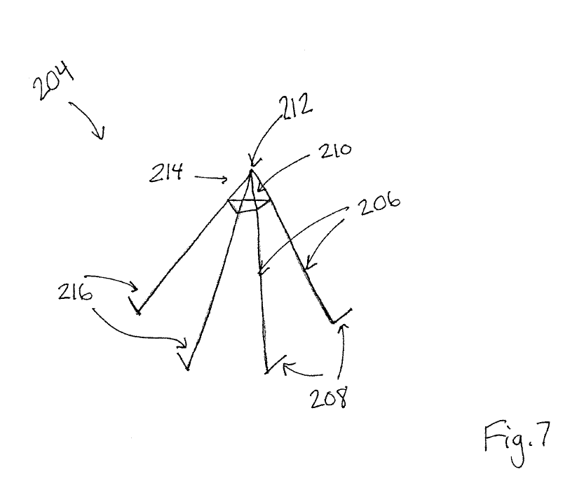

As shown in FIGS. 7-10, an alternate version of device (204) comprises legs (206) and cap (210). Each leg (206) can have a first end (214) or distal end and a second end (216) or proximal end. Device (204) may comprise any suitable number of legs (206) as will be appreciated by one of ordinary skill in the art. By way of example, device (204) may comprise a plurality of legs (206), a ring, or any other suitable attachment or structural configuration. In the illustrated version, device (204) comprises four legs (206). Legs (206) may be joined together by their first ends (214) at an apex (212) in any suitable fashion as will be apparent to those in the art. Legs (206) may be joined together, for instance, through use of an adhesive, by heat fusion, or by tying. In one version, the legs (206) are retained by a selectively changeable or removable cap (210) such that removal, dissolution, or a change in the cap (210) results in the legs (206) transitioning from an operable position to an inoperable position. The exemplary legs (206) may be joined at apex (212) by cap (210), which may be fixedly attached to or detachable from legs (206) by, for instance, an absorbable adhesive or with a material reactive to electromagnetic induction. Alternatively, the non-absorbable component may be formed around the absorbable component, so that the majority of the strand is embedded permanently within the non-absorbable strand or anchor.

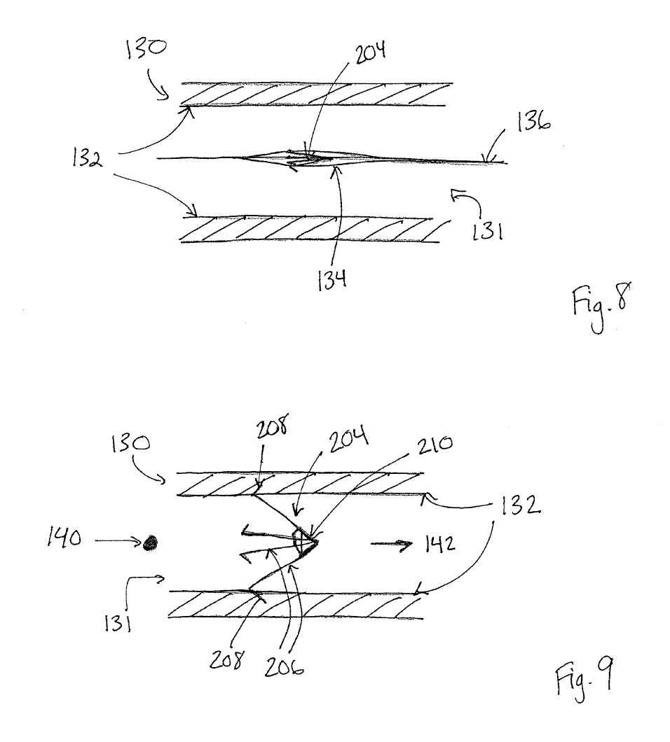

In this way, device (204) may be moveable between a collapsed position (FIG. 8) and an expanded or operable, conical position (FIG. 9). Of course, device (204) may have any other suitable structure to permit operation of the device (204) between a collapsed position and an expanded position. For example, when in the expanded or operable position, the device (204) can take on a pyramidal position. Once in the expanded position, the legs (206) can be secured to a blood vessel or other suitable physiological structure. By way of example, legs (206) may be generally planar having a webbed surface, for example, to promote ingrowth with blood vessel tissue.

At their second ends (216), legs (206) may comprise a plurality of hooks (208) or other means, member, mechanism, or feature for affixing device (204) to a blood vessel wall. Other types of fixation members that may be used in device (204) include, without limitation, low-profile fixation hooks, absorbable fixation hooks, stent fixation rings, and absorbable fixation rings. For example, device (204) may comprise a ring such as body (110) shown in FIG. 1. In the exemplary version, each leg (206) comprises one hook (208), but it will be understood that each leg (206) may comprise zero or two or more hooks (208). Hooks (208) may additionally comprise a breakpoint mechanism whereby the hooks (208) are detachable from the second ends (216) of legs (206). Hooks (208) may be detachable and/or retrievable by, for example, being joined to legs (206) via a heat-activated bonding compound that dissolves over time.

Legs (206) may also be tapered, such that they are thicker, for example, at their first ends (214) and thinner at their second ends (216). Such tapering may provide the adequate strength or other properties needed to ensure that no piece of absorbable material, such as a piece of leg (206) or adhesive, of any dangerous size that potentially breaks off or dissolves from the device (204) can be carried along by the blood flow to a potentially dangerous location, e.g., the brain, heart, or lung. Of course, legs (206) may be tapered in the opposite fashion, i.e., such that they are thicker at their second ends (216) and thinner at their first ends (214). Or legs (206) may not be tapered at all, having a consistent width along the entire length of the leg (206).

Legs (206) and/or cap (210) may be formed from any suitable biocompatible material as will be understood by one of ordinary skill in the art. For example, legs (206) (including hooks (208)) and cap (210) may be absorbable or non-absorbable. In the illustrated version, cap (210) can be formed from a bioabsorbable material, such as polydioxanone, and legs (206) may also be formed of the same material. Alternatively, legs (206) may be formed from a non-absorbable copper or other metal alloy, nickel-titanium alloy, or PTFE. Still other versions of device (204) may be made from composites of absorbable and non-absorbable materials, which may permit a controlled fragmentation and gradual absorption of the legs (206) over time to prevent a large fragment emboli from travelling to a dangerous site such as the brain or lung. Hooks (208) or other fixation means may comprise the same or different material(s) as the rest of legs (206). If a stent, for instance, is used to affix device (204) to a blood vessel, the stent may be made of a nickel-titanium alloy, copper, a bioabsorbable material, or a combination absorbable/non-absorbable composite.

Legs (206) and/or cap (210) may also be embedded or injected with a material to prevent the patient's body from rejecting the device (204) or to further impede embolus migration by expediting the dissolution of emboli or for any other reason. For example, threads legs (206) and/or cap (210) may be embedded with an anti-coagulant, such as heparin, that may be on time-control release to reduce or prevent the formation of emboli and/or expedite the dissolution of emboli. In such an example, an embolus that may be naturally absorbed by the bloodstream over a 30-90 day period, may be dissolved more quickly due to an injected material like heparin released from device (204). In another version, a radiopaque material may be incorporated into device (204) to assist in the detection of the device (204) once implanted. In another example, threads (106, 108) may be coated with a coating agent, such as a medication, for minimal release into the blood stream. Additionally, the device (204) components may be inert and non-reactive. The biomaterial and/or polymers comprising device (204) may be bonded with anticoagulants like heparin, which reduces or prevents clot formation and lengthens device patency. Other agents that could be bonded with the material are anti-platelet agents which would act to inhibit platelet aggregation, which is the first step in the clot formation process.

FIGS. 8-10 depict one example of filter device (204) positioned in the lumen (131) of a blood vessel (130), which may be defined by an interior wall (132). Device (204) may be positioned in any suitable location within the vascular system as will be appreciated by one of ordinary skill in the art. By way of example, device (204) may be placed in the inferior vena cava, the jugular vein, the brachial vein, or the common femoral vein. Device (204) may also be positioned either upstream or downstream of a junction between the hosting vein and its sizeable branches. It should be understood that placement of device (204) may be performed by any suitable known technique. For instance, while in a collapsed position as shown in FIG. 8, device (204) may be placed through the common femoral, jugular, or large-caliber peripheral veins using the Seldinger technique as is known by those in the art. In the illustrative version, device (204) is placed within a blood vessel (130) while device (204) is in a collapsed position as shown in FIG. 8. In one version, the device (204) can be positioned such that the apex (212) of the device (204) points in the direction of blood flow (142).

Once placed within the lumen (131) of blood vessel (130), device (204) may be deployed from a collapsed position to an expanded or operable position and secured to the interior wall (132) within the vessel (130) or other physiological structure. In the exemplary version, a balloon (134) and guide wire (136) may be used to selectively actuate the device (204) from a collapsed position to an expanded position. As shown in FIGS. 8-9, balloon (134), located in the interior of the collapsed device (204), may be inflated such that the inflation of balloon (134) causes a corresponding expansion of the device (204) as the second ends (216) of legs (206) move near the interior wall (132). In the expanded position, device (204) may be secured to the interior wall (132) of blood vessel (130) in any suitable manner as will be understood by one of ordinary skill in the art. For example, the expansion of balloon (134) may cause the insertion of hooks (208) into the wall (132) of the vessel (130) to secure device (204) in place. Of course, device (204) may be secured to vessel (130) in any other suitable manner as will be appreciated by one of ordinary skill in the art. By way of example, device (204) may be secured via a stent-like portion extending from the second ends (216) of legs (206). Once device (204) is in place within blood vessel (130), balloon (134) may be deflated and removed via guide wire (136).

Referring to FIG. 9, device (204) is shown in an open expanded position within blood vessel (130). In this manner, device (204) is operable to trap an embolus (140) traveling along in the direction (142) of blood flow. For example, as the embolus (140) travels along the blood stream in direction (142), it may encounter one or more legs (206) and/or cap (210) of device (204). Based on the principles of fluid dynamics, wherein blood flow is fastest near the central axis of a vessel, embolus (140) may be traveling near the center of vessel (130), such that it may contact legs (206) at a point near apex (212) and/or cap (210) where legs (206) are closely disposed. In this way, embolus (140) may be trapped and impeded from further travel by device (204). If device (204) is embedded with a drug or other material, such as an anti-coagulant, it may be released while the device (204) is positioned within the vessel (130). For example, if device (204) is embedded with a heparin anti-coagulant, the heparin may be released and dissolve an embolus (140) trapped by device (204).

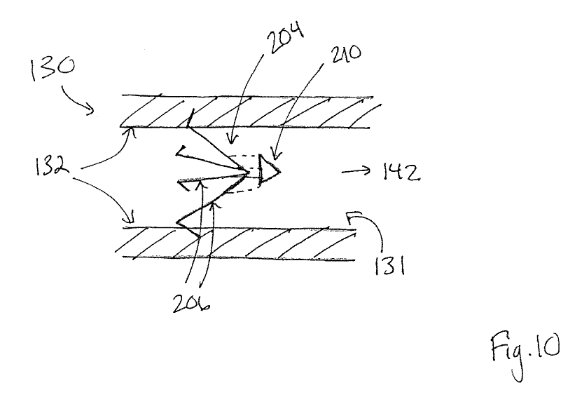

Over time, exemplary device (204) may be removed from blood vessel (130) or it may remain forever. Whether or not device (204) is removed from vessel (130) may depend on a number of factors such as the materials from which device (204) is manufactured and patient health. Even if device (204) is not actively retrieved by a surgeon or otherwise removed from blood vessel (130), it may dissolve over time such that at some point it no longer remains in the body. In some versions, device (204) may only partially dissolve, leaving at least a portion of the device (204) harmlessly where implanted in vessel (130). As depicted in FIG. 10, cap (210) may detach from apex (212) allowing the legs (206) which are secured to the blood vessel (130) to align themselves with the direction of blood flow (142) and/or the vessel wall (132), and thereafter pose no substantial impediment to blood flow. Cap (210) may detach from apex (212) because the cap itself is dissolvable, and/or because the material (e.g., adhesive) attaching the cap (210) to the apex (212) is dissolvable. In still other versions, legs (206) made at least partially from absorbable material may gradually dissolve via controlled fragmentation and/or bond dissociation. Such gradual fragmentation may prevent migration of a large emboli to a critical organ, such as the lung. Gradual absorption may also provide adequate patient protection for a predictable time period. In one version, the cap (210) is configured to dissolve or otherwise undergo a selective transformation or change including melting, deformation, dissolution, fragmentation, or any other suitable physical or chemical change, as a result of electromagnetic induction.

When selectively transformed or altered, the cap (210) may be configured to disengage from the device (204) such that the device (204) is able to transition from an operable configuration to an inoperable configuration. It will be appreciated that the any portion of the cap (210) and/or any other component of the device (204) may be configured to selectively change in response to induction to allow the filter to transition from an operable configuration to an inoperable or partially inoperable configuration. For example, a device (204) may be implanted within a portion of the vascular of a patient to provide a therapeutic effect for a period of time. At the end of this time period, when having the operable device (204), such as a vena cava filter, is no longer desirable, electromagnetic induction may be applied non-invasively outside the patient's body to cause a change in the device (204) that results in a transition from an operable configuration to an inoperable configuration. In this manner, it may be possible to render device (204) inoperable without requiring a patient to undergo an invasive procedure for the removal of the device (204) from the vasculature.

Of course, the above-described filter device (204) is merely one example. Any other suitable type of filter device (204) and associated components may be used. By way of example, legs (206) may connect planar webbed surfaces that promote ingrowth of the device (204) into a vessel wall (132). Alternatively, filter device (204) may have any other suitable components, features, configurations, functionalities, operability, etc. Other suitable variations of filter device (204) and associated components will be apparent to those of ordinary skill in the art in view of the teachings herein.

### II. Controlled Release Mechanism

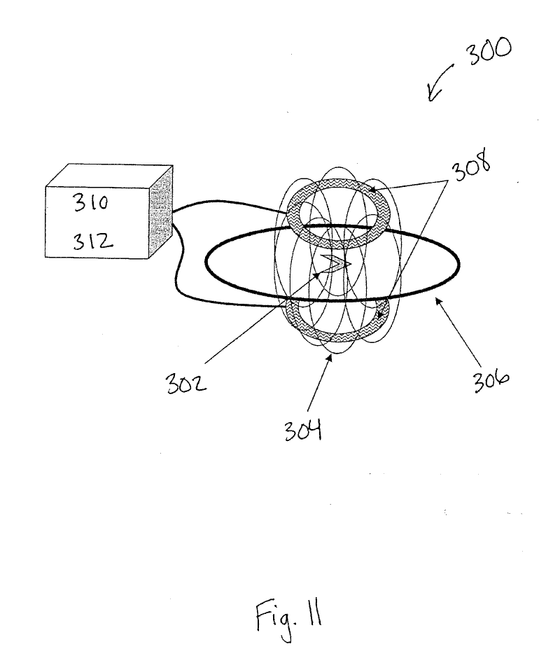

Instead of permanent implantation of a filtration device or dissolution of the device over time, a surgeon or other physician may effect removal of the device. By way of example, a surgeon may utilize an invasive surgical procedure to go in and physically remove the device. Alternatively, FIG. 11 shows one version of a system (300) that uses magnetic induction to remove a filtration device (302) in a non-invasive manner. As shown in FIG. 11, system (300) comprises a filter (302) and a magnetic field (304). Filter (302) may be positioned within the body of a patient (306) using any suitable method of insertion. In one version, the filter (302) is attached to a vessel wall or other tissue in a minimally traumatic fashion.

Filtration device (302) may be similar to the devices (104, 204) described above, or it may be another inferior vena cava filter, stent, anastomosis, or ligation device. At least a portion of filter (302) may be constructed from a material that will be affected by the magnetic field (304). By way of example, filter (302) may comprise a composite material that includes metal oxide particles as a filler in a polymer matrix. The metal oxide particles may be, for instance, iron oxide particles having an initial diameter of about 20 to about 40 nanometers. The amount of metal oxide particles used to form the filter (302) may be varied, as may the location of the filler. By way of example, the filler may be homogenously distributed throughout the polymer matrix. Alternatively, the filler may be located in a specific component or section of the filter (302).

The polymer matrix may further comprise one or more of the following exemplary polymers and/or their co-polymers: polycaprolactone (PCL), polylactic acid (PLA), ethylene vinyl acetate, polylactic glycolic acid (PGA), polymethylmythacrylate (PMMA), and a multi-block copolymer PDC that is prepared from hard segment forming poly(p-dioxanone) (PPDO), switching segment forming polycaprolactone (PCL) and 2,2(4),4-trimethylhexanediisocyanate (TMDI) as junction unit. In one version, a polycaprolactone bio-polymer is used to construct filter (302), because it is commonly available, moldable, extrudable, has an approximate 24-month hydrolysis process, and has an approximate 60 degree Celsius melt temperature that is effective to affect the filter (302) while avoiding tissue damage with a short time exposure. Other suitable combinations of plastics, metals, and/or biopolymers may be used to construct filter (302). In one version, the materials chosen to comprise filter (302) permit dynamic flexion of the filter (302) in concert with the pulsatile motion of the vessel wall or other tissue to which the filter (302) is secured.

System (300) may cause at least a portion of the filter (302) to functionally disappear or disintegrate in vivo. Such removal of the filter (302) may be caused by the application of external energy, such as a magnetic field (304), around the filter (302). The specific magnetic field (304) may be generated by, for example, a handheld device while the patient (306) is in a supine position (e.g., lying face upward). Of course, any other suitable method or configuration that permits the field (304) to penetrate the body of the patient (306) may be utilized. Other examples of devices to generate a magnetic field (304) can include a capital equipment piece and an MRI machine. An external cyclically varying magnetic field (304) may be generated by a coil (308) coupled to a controller (310) and generator (312), which together may form an induction heater. It will be appreciated by those skilled in the art that any suitable controller and generator can be used to provide a sufficient magnetic field (304). By way of example, any number, shape, and/or orientation of the coils (308) may be used to generate a sufficient magnetic field (304) strength necessary to penetrate the patient (306). For example, system (300) may comprise only a single coil (308) that circumferentially surrounds a patient's (306) body. Alternatively, and as depicted in FIG. 11, system (300) may comprise two opposed generally planar (e.g., pancake) coils that are positioned on either side of the patient (306). In still another example, system (300) may comprise four coils (308) surrounding the patient (306) every 90 degrees. In sum, system (300) may comprise any device producing a fluctuating magnetic field of sufficient strength at a high enough frequency to cause heating of the metal or metal oxides in the filter device (302).

The magnetic field (304) generated causes the heating of the metal oxide particles within the filter (302). By “heating” it is meant that thermal energy is used to create a controlled structural failure or transformation in the filter (302) by causing the filter (302) to, for example, melt, soften, be rendered structurally insufficient, degrade, and/or disintegrate, thus rendering the filter (302) inoperable. By way of example, heating of the filter (302) inside the patient (306) may cause the filter (302) to change phase, it may cause thermal expansion of a crack in the filter (302), and/or it may cause a shape memory effect. For example, if the filter (302) comprises a shape memory nickel-titanium alloy or a shape memory plastic, the application of thermal energy to the filter (302) may cause the filter (302) to change shape and/or expand, which may then cause the filter (302) to fail structurally by, for example, de-latching from a blood vessel wall. In one version, if the filter (302) is at least partially constructed out of a more brittle material, such as polymethylmythacrylate (PMMA) or bone cement, the application of thermal energy to the filter (302) may cause expansion of a crack in the filter (302) that renders it structurally insufficient for its purpose.

Specifically, heating of the metal oxide particles which at least in part form the filter (302) may cause the melting or softening of the surrounding polymer matrix. When the polymer matrix of filter (302) is softened sufficiently, its structural integrity may be weakened, which allows at least a portion of the filter (302) to disintegrate and/or be passed through the patient's body for evacuation. By way of example, if filter (302) particles disintegrate to a particle diameter in the range of about 6 to about 15 nanometers, the particles may be small enough for removal through extravasations or renal clearance. Alternatively, disintegrated filter (302) particles may be incorporated into a vessel wall of the patient (306).

As mentioned previously, the application of the magnetic field (304) may cause total or at least partial disintegration of the filter (302). In some versions, where the metal oxide filler of filter (302) is located in a cap or other distinct spot of filter (302), the application of the magnetic field (304) may cause just the cap to melt away. In this way, if system (300) comprises the filter (204) as illustrated in FIG. 7-10, cap (210) may be melted away, and the remaining filter components may be released. The application of the magnetic field (304) at multiple different frequencies may cause multiple effects in the filter (302), because a specific frequency induces a specific amount of heat or change. For example, if a filter (302) is comprised of at least two different materials, the field (304) at a first frequency may cause the first material to undergo a structural and/or phase change, while application of the field (304) at a second frequency may cause the second material to undergo a structural and/or phase change. In this way, system (300) may cause the cap of a filter, constructed from the first material, to release separately from the release of the body of the filter, constructed from the second material. Such a capability may be used when attaching or deploying a filter (302). For example, release of a cap separately may cause the blood flow in the vessel to force the body of the filter up against the vessel wall, thereby rendering the filter functionally useless. The amount of field strength and frequency needed to create a controlled structural failure in filter (302) may be affected by the shape of filter (302), amount of material the filter (302) is constructed from, and/or the surface to volume ratio of the filter (302).

In other versions of system (300), the application of a specific magnetic field (304) strength to the filter (302), may cause one or more effects in the filter (302) that serve the same purpose. For example, at a single frequency, the magnetic field (304) may cause the cap of the device (302) to release, the filter (302) to change shape, and the body of the filter (302) to actively bias outward to the vessel wall. In this way, redundancy is built into the system (300) such that degradation of the material comprising filter (302) does not have to be relied upon to cause the structural failure of the device (302).

In still another version, system (300) may be used to induce only one physical change in the filter (302). Other methodologies, such as ultrasound, high frequency, radio frequency ablation, or an invasive surgical technique, could be used to perform any other changes to the filter (302). By way of example, a magnetic field (304) may be used to cause the cap of the filter (302) to release, and then a surgeon could perform an intravascular procedure to manually remove the body of the filter (302) if desired. Alternatively, a surgeon could initially remove the cap of a filter (302) invasively and then use the system (300) to induce a magnetic field (304) to cause the body of the filter (302) to disintegrate. In this way, it may be relatively easier to remove the filter (302) completely from a vena cava or other blood vessel. In an alternate version, a small sheath could be inserted into the blood vessel wall and a wire guided into a vessel to release the cap. So even if a magnetic field (304) is not generated or cannot be generated, filters may be provided with a fail-safe method for removing all or a portion of the filter (302).

Of course, the above-described induction system (300) is merely one example. Any other suitable type of system (300), configuration, and associated components may be used. By way of example, in addition to providing the energy that causes at least a portion of filter (302) to disintegrate, magnetic field (304) and coils (308) may be used to help detect the location of the filter (302) within the body of the patient (306). Precise locating of the filter (302) may increase the likelihood that effective treatment is provided.

System (300) may give doctors the confidence to know they can place a filter (302) and subsequently “turn it off” or disable the filter non-invasively. In this way, patient care may shift to a simple office visit, which may increase the likelihood of follow-up. And with filters (302) no longer functionally in place, any long-term complications and costs associated with current designs and procedures may be reduced or eliminated. System (300) may also eliminate any complications posed by follow-up removal via invasive techniques.

It should be understood that any feature(s) and/or operability described herein with respect to one particular filtration device (104, 204, 302) may be incorporated into any other filtration device (104, 204, 302) described herein. By way of example only, any of filtration devices (104, 204, 302) may be modified to include a cap (210). Similarly, any filtration device (104, 204, 302) described herein may be used in accordance with the exemplary uses taught herein with respect to one particular filtration device (104, 204, 302) described herein. Therefore, none of the teachings herein should be understood as applying to only one particular version or embodiment of filtration device (104, 204, 302) described herein. Every teaching herein is contemplated as being interchangeable among versions and embodiments, such that every teaching herein may be applied to any filtration device (104, 204, 302) described herein. Various ways in which the teachings herein may be interchanged among various versions and embodiments will be apparent to those of ordinary skill in the art in view of the teachings herein.

As noted above, the filters (104, 204, 302) and induction system (300) described herein may be used for the prevention of pulmonary embolism and deep vein thrombosis. Of course, the filters (104, 204, 302) and system (300) may be used in conjunction with any other suitable surgical or medical procedure, treatment, or specialty, such as trauma, bariatrics, oncology, or orthopedics. For example, system (300) may be applicable to areas such as peripheral vascular and cardiovascular stents, staples, anastomotic devices used in vascular and gastrointestinal surgery, bariatric devices used for weight loss, etc. Still other various settings and combinations in which a filtration device (104, 204, 302) and system (300) may be used will be apparent to those of ordinary skill in the art in view of the teachings herein.

While several filtration devices have been discussed in detail above, it should be understood that the components, features, configurations, and methods of using the filtration devices discussed are not limited to the contexts provided above. In particular, components, features, configurations, and methods of use described in the context of one of the filtration devices may be incorporated into any of the other filtration devices. For example, device (104) may have a cap (210) that is detachably attached to the superior apex (116). In another example, instead of hooks (208), device (204) may comprise a body (110) at the second ends (216) of legs (206).

FIGS. 12-14 illustrate alternative embodiments of filtration devices or filters configured for placement within a portion of the vasculature of patient or within any other suitable physiological structure. The filtration devices include releasable caps configured for attachment to a plurality of filter or thread elements, where the cap is configured to release, for example, after being acted upon by electromagnetic induction. The caps or any other elements of the filter may, for example, include metal oxide particles that respond to electromagnetic induction such that the cap is released from the filter elements to transition the filter from an operable configuration to a substantially inoperable configuration.

Having shown and described various versions in the present disclosure, further adaptations of the methods and systems described herein may be accomplished by appropriate modifications by one of ordinary skill in the art without departing from the scope of the present application. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present application should be considered in terms of the following claims and is understood not to be limited to the details of structure and operation shown and described in the specification and drawings.

What is claimed is:

1. A filtration device comprising:
a. a body configured for placement within a portion of the vasculature of a patient, wherein the body comprises a plurality of threads, each thread having a proximal end and a distal end;
b. an cap forming an apex, wherein at least one of the cap and the body comprises a selectively transformative material;
c. a first configuration, wherein the cap substantially retains the distal end for each of the plurality of threads at the apex such that the filter device is substantially operable; and
d. a second configuration, wherein the filtration device is substantially inoperable as a result of at least one of the body and the cap being selectively transformed by electromagnetic induction.

2. The filtration device of claim 1, wherein at least one of the cap and the body undergo at least a physical transformation or a chemical transfoimation in the second configuration.

3. The filtration device of claim 1, wherein the cap is configured to be releasable.

4. The filtration device of claim 3, wherein the body is configured to transition from the first configuration to the second configuration upon release of the cap from the body.

5. The filtration device of claim 1, further comprising at least one vessel attachment member associated with the plurality of threads, wherein the at least one vessel attachment member is configured to secure the body to a portion of the vasculature of the patient.

6. The filtration device of claim 1, wherein at least a portion of the plurality of threads comprises a dissolvable material.

7. The filtration device of claim 1, wherein the plurality of threads comprises an agent selected from the group consisting of an anti-coagulant agent, an anti-platelet agent, a coating, a medication, and combinations thereof.

8. The filtration device of claim 1, wherein the body is selectively transitioned between a closed configuration for insertion of the body into a portion of the vasculature of the patient and an open configuration for securing the body to the vasculature of the patent, wherein the width of the body when in the open configuration is greater than the width of the body when in the closed configuration.

9. The filtration device of claim 1, wherein at least a portion of at least one of the cap and the body comprises a transformative material, the transformative material comprising metal oxide particles.

10. A filtration system comprising:
a. a filtration device comprising;
i. a body configured for placement within a portion of the vasculature of a patient, wherein the body comprises a plurality of threads, each thread having a proximal end and a distal end;
ii an cap forming an apex, wherein at least one of the cap and the body comprises a selectively transformative material;
iii. a first configuration, wherein the cap substantially retains the distal end for each of the plurality of threads at the apex such that the filtration device is substantially operable; and
iv. a second configuration, wherein the filtration device is substantially inoperable as a result of at least one of the body and the cap being selectively transformed by electromagnetic induction; and
b. an electromagnetic induction system, the electromagnetic induction system being configured to generate a magnetic field sufficient to selectively transform the filtration device from the first configuration to the second configuration.

11. The filtration system of claim 10, wherein the electromagnetic induction system comprises a handheld device.

12. The filtration system of claim 10, wherein the electromagnetic induction system comprises a coil coupled to a controller and a generator configured to generate the magnetic field.

13. The filtration system of claim 10, wherein the electromagnetic induction system is configured to selectively provide the magnetic field at a first frequency and at a second frequency.

14. The filtration device of claim 10, wherein at least one of the cap and the body undergo at least a physical transformation or a chemical transformation in the second configuration.

15. A filtration method comprising the steps of:
providing a filtration device comprising;
a. a body configured for placement within a portion of the vasculature of a patient, wherein the body comprises a plurality of threads, each thread having a proximal end and a distal end;
b. an cap forming an apex, wherein at least one of the cap and the body comprises a selectively transformative material;
c. a first configuration, wherein the cap substantially retains the distal end for each of the plurality of threads at the apex such that the filtration device is substantially operable; and
d. a second configuration, wherein the filtration device is substantially inoperable as a result of at least one of the body and the cap being selectively transformed by electromagnetic induction;
providing an electromagnetic induction system;
inserting the filtration device into a portion of the vasculature of a patient; and
generating a magnetic field with the electromagnetic induction system to selectively transform the filtration device from the first configuration to the second configuration.

16. The filtration method of claim 15, wherein the step of inserting the filtration device into the vasculature of the patient further comprises inserting the filtration device in a reduced diameter configuration and selectively expanding the filtration device to engage the vasculature of the patient.

17. The filtration method of claim 15, wherein the step of generating a magnetic field with the electromagnetic induction system is performed non-invasively.

18. The filtration method of claim 15, wherein the step of generating a magnetic field with the electromagnetic induction system comprises generating the magnetic field at a first frequency to provide a first effect and at a second frequency to provide a second effect.

19. The filtration method of claim 15, wherein the electromagnetic induction system is a handheld device.

20. The filtration method of claim 15, wherein at least a portion of the filtration device is selectively dissolvable.
